(19) United States
(12) Patent Application Publication
(10) Pub. No.: US 2018/0264321 A1
(43) Pub. Date: Sep. 20, 2018

(54) TILTING SURFACE APPLICATION CONTROLLER

(71) Applicant: BO & BO LTD., Mosshav Tzofit (IL)

(72) Inventors: Gadi Nir, Moshav Tzofit (IL); Eitan Merhavi, Shaeri Tikva (IL)

(21) Appl. No.: 15/537,606

(22) PCT Filed: Dec. 14, 2015
(86) PCT No.: PCT/IL2015/051211

§ 371 Date: Jun. 19, 2017

Related U.S. Application Data
(60) Provisional application No. 62/092,854, filed on Dec. 17, 2014.

(51) Int. Cl. A63B 24/00; A63B 26/00; A63B 22/16; A63B 22/18; A63B 5/11; A61B 5/11; A63F 13/211; A63F 13/816

(52) U.S. Cl. CPC A63B 24/0087; H04M 1/04; A63B 22/16; A63B 22/18; A63B 5/11; A63B 24/0062; A61B 5/11; A63F 13/211; A63F 13/816; A63B 2024/0096; A63B 2208/0204; A63B 2220/44; A63B 2225/52; A61B 2503/12; A61B 2562/0219; H04M 2250/12; A63B 26/003

(57) ABSTRACT

The present invention relates to an electronic device configured to be fixed to a standing surface; wherein said electronic device comprises: a processor; a sensor, wherein said sensor is selected from one of the following sensors: a. an accelerometer sensor configured to estimate the acceleration of said standing surface and provide acceleration data; and wherein said processor is configured to receive said acceleration data from said accelerometer sensor and convert said accelerometer data into corresponding generic key stroke commands of a standard keyboard; b. a gyroscope sensor configured to estimate angular data of said standing surface and provide angular data; and wherein said processor is configured to receive said angular data from said gyroscope sensor and convert said angular data into corresponding generic key stroke commands of a standard keyboard; a transmitter configured to transmit said generic key stroke commands to a remote device; and a power source.

### FIELD OF THE INVENTION

This invention relates to the fields of physiotherapy, exercise, fitness, and gaming. In particular, this invention relates to a device used for balance exercises.

### BACKGROUND

Nowadays, there are many methods for balance training. These methods are fully distributed in physiotherapy facilities, such as private or public institutes, rehabilitation institutions, neurological institutions, child development institutions, geriatric institutions, and hospitals. There is also a growing distribution of balance training in the field of sports and leisure, gyms, and in private homes.

The classic methods of unstable balance surfaces are divided into three categories:
1. Methods for balance training on a single axis such as the half foam roller, e.g. GFROLL by General Fitness, catalog no: 221625.
2. Training methods on more than one axis such as the Balance Board and BOSU®, for example the BOSU® Balance Trainer, the balance board—Sissel® Balance Board.
3. Training methods without an axis, based on air pressure changes, such as the balance disc. For example, the AVITA balance disc, catalog no. 59180.

Generally speaking, unstable balance surfaces used in balance training are characterized by one degree of freedom or more, and allow axial movement. Usually the person undergoing the training stands on the surface, and tries to maintain stability without falling. In addition, exercises and activities can be combined while maintaining stability on the surface. Training on surfaces is designed to improve balance and strengthen the core muscles, and has been shown in many studies to be very effective in the rehabilitation and prevention of injuries such as sports injuries, falling of elderly people, and more.

“Effective Exercise for the Prevention of Falls: A Systematic Review and Meta-Analysis”, (Catherine Sherrington PhD, Julie C. Whitney MSc, Stephen R. Lord DSc, Robert D. Herbert PhD, Robert G. Cumming PhD and Jacqueline C. T. Close MD; Article first published online: 31 Oct. 2008 Journal of the American Geriatrics Society; Volume 56, Issue 12, pages 2234-2243, December 2008) is an article that relates to determining the effects of exercise on falls prevention in older people and establish whether particular trial characteristics or components of exercise programs are associated with larger reductions in falls.

Neuromuscular training for sports injury prevention: a systematic review (Hübscher M, Zech A, Pfeifer K, Hansel F, Vogt L, Banzer W; Department of Sports Medicine, Goethe-University Frankfurt, Frankfurt, Germany, Medicine and Science in Sports and Exercise Journal [2010, 42(3):413-421]), is an article that relates to assessing the effectiveness of proprioceptive/neuromuscular training in preventing sports injuries by using the best available evidence from methodologically well-conducted randomized controlled trials and controlled clinical trials without randomization.

Use of an interactive video gaming program compared with conventional physiotherapy for hospitalised older adults: a feasibility trial (Kate Laver, Stacey George, Julie Ratcliffe, Steve Quinn, Craig Whitehead, Owen Davies, and Maria Crotty; Flinders University Department of Rehabilitation and Aged Care, Adelaide, South Australia; October 2012, Vol. 34, No. 21, Pages 1802-1808 (doi:10.3109/09638288.2012.662570)) is an article that relates to assessing the feasibility of a physiotherapy intervention using an interactive gaming program compared with conventional physiotherapy for hospitalized older people.

However, the prior art still lacks efficient means for providing a fun, easy to use exercise with high interest and with providing the required medical exercise application. It is therefore an object of the present invention to provide a method and means for efficient physiotherapy exercises

It is a further object of the present invention to provide a method and means for fun physiotherapy exercise games accessible to users having smart mobile devices.

It is a further object of the present invention to provide a method and means for fun physiotherapy exercise games accessible to users on a device mountable on several different bases/platforms.

Other objects and advantages of the present invention will become apparent as the description proceeds.

### SUMMARY OF THE INVENTION

The present invention relates to a system with an alterable platform such that altering the platform directly causes a change in the function of a running computer application. The change in the function is preferably proportional to the altering of the platform. For example, when tilting the platform, the speed of the tilt, the magnitude of the tilt, the radial direction of the tilt, the acceleration of the tilt, etc., influence a proportional change in a suitable running application. Thus the present invention can contribute to many physiotherapy applications such that when the platform is a tiltable standing surface, dedicated computer applications can enable influence of the running application due to required physiotherapy tilt exercises. Preferably, the running application is a fun game encouraging a user patient to apply certain exercises in order to succeed in the game. The present invention can also be used for medical physiotherapy evaluation, analysis and follow-up.

The present invention can be used with existing therapy platforms (e.g. a BOSU device) wherein a slight moving of the platform directly affects the running application. The platform preferably comprises acceleration and gyroscopic sensors that deliver online data to a processor running the application.

According to a preferred embodiment, the platform is a mountable standing surface, mountable on existing therapy platforms or other receiving platforms. The mountable standing surface comprises acceleration and gyroscopic sensors that deliver online data to a processor running the application. In a preferred embodiment, the acceleration and gyroscopic sensors are part of an electronic device such as a mobile device. The mobile device is fixable on/within the mountable standing surface. Preferably, the mobile device comprises a processor/operating system, which can run the applications. Nowadays, wherein several people own a mobile device, using the present invention can be accessible to many people.

Preferably, display means receive the running application data such that the running application can be viewed on the display means. Optionally, sound making (or noise making) devices can produce various sounds instead of (or in addition to) the display means such that a slight moving of the platform directly affects the running application and thus the sound effect produced by the sound making devices.

The present invention is not limited to specific exercises, but can be used by various users (e.g. playing a tilting game for fun).

The present invention relates to a system comprising a tiltable platform, comprising:
a. an acceleration or gyroscopic sensor;
b. transmitting means for transmitting acceleration or gyroscopic data received from the acceleration or gyroscopic sensors;
   said system further comprising receiving means for receiving said acceleration or gyroscopic data; a processor, capable of running an application, configured to receive said acceleration or gyroscopic data from said receiver; and display means connected to said processor.

The present invention relates to a system comprising a mountable standing surface configured to receive a mobile device being fixed thereto in a recess within said mountable standing surface.

The present invention relates to an electronic device configured to be fixed to a standing surface; wherein said electronic device comprises:
a processor;
a sensor, wherein said sensor is selected from one of the following sensors:
   a. an accelerometer sensor configured to estimate the acceleration of said standing surface and provide acceleration data; and wherein said processor is configured to receive said acceleration data from said accelerometer sensor and convert said accelerometer data into corresponding generic key stroke commands of a standard keyboard;
   b. a gyroscope sensor configured to estimate angular data of said standing surface and provide angular data; and wherein said processor is configured to receive said angular data from said gyroscope sensor and convert said angular data into corresponding generic key stroke commands of a standard keyboard;
a transmitter configured to transmit said generic key stroke commands to a remote device; and
a power source.

Preferably, the standing surface is a mountable standing surface.

Preferably, the corresponding generic key stroke commands of a standard keyboard are the arrow keys.

The present invention relates to an electronic device configured to be fixed to a standing surface; wherein said electronic device comprises:

a processor;
a sensor, wherein said sensor is selected from one of the following sensors:
a. an accelerometer sensor configured to estimate the acceleration of said standing surface and provide acceleration data; and wherein said processor is configured to receive said acceleration data from said accelerometer sensor and convert said accelerometer data into corresponding generic key stroke commands of a standard keyboard;
b. a gyroscope sensor configured to estimate angular data of said standing surface and provide angular data; and wherein said processor is configured to receive said angular data from said gyroscope sensor and convert said angular data into corresponding generic key stroke commands of a standard keyboard;

a cable configured to connect to a remote device, and configured to transfer said generic key stroke commands to a remote device; and a power source.

The present invention relates to a system comprising:
a. a mountable standing surface configured to be mounted and fixed to the standing surface of an unstable balance surface;
b. the electronic device according to claim 1 or 4, wherein said electronic device is fixed to said mountable standing surface.

The present invention relates to a method for controlling an application running on an operating system, according to the tilts of a standing surface, said method comprises the following steps:

providing a processor and a sensor,
a. obtaining accelerometer or gyroscope data from said sensor;
b. transferring said accelerometer or gyroscope data to a processor;
c. converting said accelerometer or gyroscope data into corresponding keyboard commands by said processor;
d. transmitting said keyboard commands to a remote device having a second processor running an application on an operating system;
e. transferring the transmitted keyboard commands to said running application to be controlled accordingly.

The present invention relates to a system comprising:
a. a mountable standing surface configured to be mounted and fixed to the standing surface of an unstable balance surface;
b. an electronic device wherein said electronic device is fixed to said mountable standing surface and said electronic device comprises:
   I. an accelerometer sensor or gyroscope sensor configured to provide accelerometer sensor data or gyroscope sensor data;
   II. a processor running an application, wherein said running application is configured to receive said accelerometer sensor data or gyroscope sensor data and said running application is controlled according to said accelerometer sensor data or gyroscope sensor data.

Preferably, the electronic device is a mobile device.

Preferably, the system further comprises a transmitter configured to transmit the running application to a remote device with a screen.

Preferably, the system further comprises a cable configured to transmit the running application to a remote device with a screen.

The present invention relates to a system comprising:
a. a mountable standing surface configured to be mounted and fixed to the standing surface of an unstable balance surface;
b. an electronic device wherein said electronic device is fixed to said mountable standing surface and said electronic device comprises:
   I. an accelerometer sensor or gyroscope sensor configured to provide accelerometer sensor data or gyroscope sensor data;
   II. a transmitter configured to receive said accelerometer sensor data or gyroscope sensor data and transmit said accelerometer sensor data or gyroscope sensor data, to a remote device with a processor running an application, for controlling said running application.

Preferably, the electronic device is a mobile device.

The present invention relates to a system comprising:
a. a mountable standing surface configured to be mounted and fixed to the standing surface of an unstable balance surface;
b. an electronic device wherein said electronic device is fixed to said mountable standing surface and said electronic device comprises:
   I. an accelerometer sensor or gyroscope sensor configured to provide accelerometer sensor data or gyroscope sensor data;
   II. a cable configured to transfer said accelerometer sensor data or gyroscope sensor data, to a remote device with a processor running an application, for controlling said running application.

The present invention relates to a mountable standing surface configured to be mounted and fixed to the standing surface of an unstable balance surface; wherein said mountable standing surface comprises a recess to receive an electronic device.

### BRIEF DESCRIPTION OF THE DRAWING

The present invention is illustrated by way of example in the accompanying drawings, in which similar references consistently indicate similar elements and in which:

FIG. 1 illustrates an embodiment of the mountable standing surface.

FIG. 2A-2B illustrate another embodiment of the mountable standing surface.

FIG. 3A illustrates an embodiment of the mountable standing surface before being mounted on a BOSU device.

FIG. 3B illustrates an embodiment of the bottom portion of the mountable standing surface

FIG. 4 illustrates an embodiment of the mountable standing surface before being mounted on a balance disc.

FIG. 5 illustrates an embodiment of the mountable standing surface before being mounted on a bagel balance disc.

FIG. 6 illustrates an embodiment of the mountable standing surface before being mounted on a half foam roller.

FIGS. 7A-7C illustrate embodiments of the mountable standing surface mounted on pillows, a trampoline and an upside down BOSU device, respectively.

FIG. 8A-8C illustrate embodiments of the mountable standing surface mounted on a BOSU device wherein the user is playing across from a screen.

FIG. 9 illustrates an example of a running application game played with the present invention.

FIGS. 10a-10c illustrate an example of a running application game played with the present invention.

FIG. 11 illustrates a block diagram of a preferred embodiment.

FIGS. 12a-12b illustrate an embodiment of the mountable standing surface of the present invention.

FIGS. 13a-13b illustrate an embodiment of the mountable standing surface of the present invention.

FIG. 14 illustrates an embodiment of the mountable standing surface of the present invention.

FIG. 15. Illustrates embodiments of the mountable standing surface of the present invention fixed to an unstable balance surface

FIG. 16. Illustrates the method steps of an embodiment of the present invention.

### DETAILED DESCRIPTION OF THE PREFERRED EMBODIMENT

The present invention relates to a system that enables a synergetic interface between an electric/electronic device (e.g. a mobile device, such as a smart phone, PDA, handheld computer or tablet) and unstable balance surfaces. The unstable balance surfaces comprise a standing surface for the user to stand on, configured to connect to the base portion of the unstable balance surfaces. The standing surface comprises means for receiving and protecting the electric/electronic device (e.g. mobile device) and for fixing it thereto. The interaction between these elements enables various physiotherapy treatment applications.

According to a preferred embodiment, the unstable balance surface comprises two main components:
(a) The standing surface—the upper part of the surface on which the user's limbs are placed, which control the movement of the surface by shifting weight to a certain portion.
(b) The base of the surface—The part located under the Standing surface and which makes contact with the floor and which is used as an axis or base for the movement of the Standing Surface. The base of the surface is typically round (preferably spherical) at its bottom enabling tilting the unstable balance surface to any required direction (360 degrees).

Examples of balance surface boards that can be used in the system of the present invention are the balance board—Sissel® Balance Board, a half foam roller, e.g. GFROLL by General Fitness catalog no: 221625, the AVITA balance disc catalog no. 59180, the BOSU® Balance Trainer.

According to a preferred embodiment, the mobile devices comprise gyroscope and/or accelerometer sensors. Advanced mobile devices such as the Nexus 7 2013, the iPhone, iPad, and Galaxy etc., contain built-in gyroscope and accelerometer sensors. These types of sensors contribute to the operation of many applications and games.

The mobile device is fixed to the standing surface, thus they tilt together as a unit. The mobile devices comprise gyroscope and/or accelerometer sensor-based applications. The gyroscope sensor is configured to provide gyroscope data that includes the relative changes in the sensor angle. Also the gyroscope sensor is configured to provide location data including longitude, latitude and altitude data. The accelerometer sensor is configured to provide acceleration data that includes the relative changes in the sensor acceleration. Also the accelerometer sensor is configured to provide location data including longitude, latitude and altitude data.

The mobile device comprises a processor that is configured to accept the data received from the gyroscope and accelerometer sensors. The processor is configured to run applications that receive the gyroscope and accelerometer sensors data (e.g. location data) and respond accordingly. The application may be a real time game, a real time information display, and other sensor based application that responds according to the movement of the gyroscope and accelerometer sensors, which move according to the movement (tilt) of the surface. As a result, one can control applications and games through the movement of the body on the surface, by shifting weight in different directions, and causing a tilt accordingly.

The present invention is particularly effective when using any kind of professional training and treatment unstable balance surfaces (along with the elements of the present invention), that have proven their effectiveness in therapy, improving balance and preventing injuries and falls.

According to one embodiment, the standing surface is part of a total unit with the base portion beneath the standing surface. According to a preferred embodiment, the present invention relates to a mountable standing surface, which is typically mounted onto an existing standing surface of an unstable balance surface, but could also be mounted on other surfaces that enable tilt of the mountable standing surface, such as a trampoline, or a pillow. The mountable standing surface comprises a space into which the mobile device (or electric device comprising the sensors) is configured to be inserted thereinto and fixed thereto.

According to one embodiment, the system comprises mobile devices that do not necessarily comprise gyroscope and accelerometer sensors. According to this embodiment, the unstable balance surface standing surface (or the aforementioned mountable standing surface) comprise insertable units fixed thereto, comprising gyroscope and/or accelerometer sensors and transmitting means based on wireless technology standard for exchanging data over short distances. The insertable units transmitting means (e.g. Bluetooth, WIFI, RF, other known wireless based communication protocols) transmit the gyroscopic/acceleration based data from the sensors to the mobile device, in real-time. The mobile devices comprise appropriate receiving means accordingly. According to another embodiment, these insertable units can transmit the data in real time by means of a wire connection to the mobile devices.

According to another embodiment the unstable balance surface standing surface (or the aforementioned mountable standing surface) comprises insertable units, comprising gyroscope and/or accelerometer sensors, fixed thereto. The insertable units comprise transmitting means based on wireless technology standard for exchanging data over short distances. The insertable units transmit the data directly to a receiving displaying device such as a smart TV or a PC, without the need of an intermediate mobile device. The insertable units can transmit the data directly to any receiving displaying device with an appropriate processer (running an appropriate present invention application) and display means. The receiving displaying device (e.g. smart TV, PC) comprise appropriate receiving means accordingly. For example, a specific smart TV comprises an insertable unit comprising receiving means and a processor. An example of such unit is the Dongle InfraBit. RK3188 1.6 GHZ QUADCORE ANDROID. According to another embodiment, these insertable units can transmit the data in real time by means of a wire connection to the receiving displaying device.

The height of the insertable unit is usually between 1.5 cm and 2 cm. The length of the insertable unit is usually between 3.3 cm and 7 cm. The width of the insertable unit is usually between 2 cm and 4 cm. The unstable balance surface standing surface (or mountable standing surface) comprise recesses with sizes adequate to receive the insertable units. The insertable unit are fixed therewithin.

When using the embodiment with the mobile device, the applications that run in real time comprise an appropriate display for the user. The appropriate display is typically displayed on the present invention mobile device screen. The present invention system mobile devices typically comprise wireless transmitting means, e.g. mirroring technologies such as Apple's Air Server, Miracast, Bluetooth, WIFI, RF, etc. or other known wireless based communication protocols. These transmitting means are configured to transmit the display data (configured to be displayed on the mobile device display screen) to an external display means such as a smart TV, TV BOX screen (such as “Roku”, Minix neo”, “Nexus player”, “Fire tv” etc.) or another external screen such as a PC screen, or a projector that projects the display data onto a surface (e.g. a projection screen). The external display means that receive the data comprise compatible receiving means (e.g. receiver). Optionally, the connection to the external display means may be by a wire connection (e.g. cables such as “Slimport” and “MHL”). The external display means display the running applications (e.g. games) in real-time and it is comfortable to operate the application when viewing the external display means. For example, a user may play a tilt based game when standing on a mountable surface at a distance of one and a half meters from a giant TV screen. This contributes to the game experience.

When the mobile device is inserted into the standing surface (or mountable standing surface) and the user stands on it, the mobile device controlling means become unreachable. Controlling the mobile device (e.g. choosing and activating a requested application) cannot be done by the mobile device input buttons or touch-screen, due to the fact that it is inserted into the standing surface. The controlling of the mobile device can be implemented by using an external wireless based (e.g. via Bluetooth) controlling implement such as a mouse, joystick, keyboard, etc. that enables controlling the mobile device. The mobile device comprises appropriate receiving means (e.g. receiver) accordingly. Optionally, the controlling implement can be connected to the mobile device by wire means. Preferably, a surface (e.g. table) for receiving the controlling implement (e.g. mouse) thereon is placed near the standing surface at a height compatible for the user hands. Optionally, the surface is placed distantly from the user, and the user can use the controlling implement to activate an application and just walk to the standing surface and begin controlling the application, or a distant personnel (e.g. physiotherapist) can activate the application using the controlling implement distantly. Another option is that the user can use his hip (or other body part) as a receiving surface to the wireless controlling implement. In the embodiment of the insertable units that transmitting data to a TV box or smart TV, one can use the TV box or smart TV original remote controller. Also, the controlling implements can be used in conjunction with the tilting in a combined application applying tilt and using a joystick, mouse, wireless controller, etc.

The synergetic connection opens up a new and useful world of possibilities for the fields of clinical and leisure sports. It allows clinicians to work with patients without having to be next to them thus saving on human resources. It enables patients to have versatile training, motivating them and improving the quality and duration of the treatment. It enables diagnostic applications to be run. Preferably, the mobile device comprises a memory for saving the users surface tilt data (and/or comprise means for transmitting it to an external factor (an external memory). Medical personnel can review and analyze this data and treat the user accordingly.

In the field of sport and quality of life, it enables every amateur or professional athlete to improve motor control and balance in a challenging and independent way. The training according to dedicated applications can assist in lowering the risk of sports injuries and the risk of falls among adults. As a leisure tool, it enables combining the pleasure of gaming with the added benefit of improving balance and motor skills.

Another advantage is due to the accessibility of mobile devices to the public. For example, one user can insert his smart phone into the standing surface (or mountable standing surface) and play a certain application game. When he finishes, he merely pulls out the mobile device and leaves. The next user that arrives can insert his mobile device in the standing surface, wherein his mobile device comprises his own applications.

The figures below illustrate the configurations and components of the present invention.

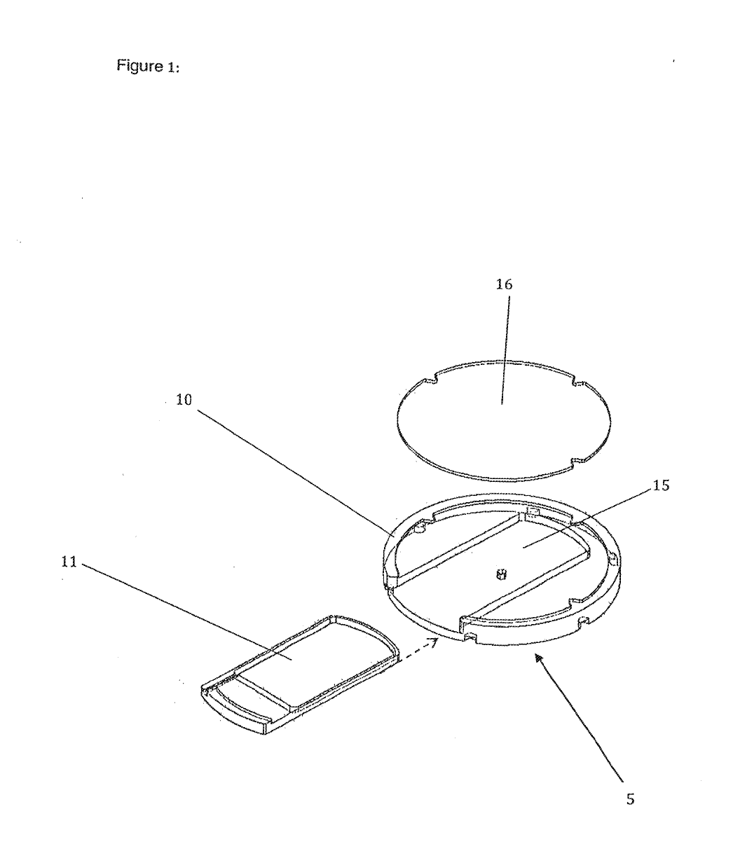

FIG. 1 illustrates the embodiment of the present invention comprising a mountable standing surface 5, which is mounted onto an existing standing surface of an unstable balance surface. The mountable standing surface 5 comprises a main body portion 10 comprising a circular shape having a diameter usually between 30-40 cm, and preferably 40 cm. the main body portion 10 diameter is usually between 15-40 cm. The Standing Surface main body portion 10 is preferably made of material selected from the group consisting of wood, plastic and metal.

The main body portion 10 comprises a modular drawer mechanism. The main body portion 10 comprises a slidable drawer 11 configured to be inserted into a recess 15 (an indentation) within the main body portion 10. The length of drawer 11 is usually between 10-20 cm. Its width is usually between 10-15 cm. Its thickness is usually between 1-4 cm. A system mobile device is configured to be mounted and fixed on drawer 11. The recess 15 size is configured to receive the drawer with the mobile device mounted/fixed thereon. The recess 15 size is such that it is compatible to receive the drawer 11.

The drawer 11 is preferably made of material selected from the group consisting of wood, plastic and metal. The fixation of the mobile device to the drawer 11 is preferably implemented as follows. According to one embodiment, drawer 11 comprises a friction surface at its top portion. The friction surface prevents the mobile device from moving to the sides (or forward/backwards). According to another embodiment, a magnet is attached to the back of the mobile device and another magnet attached to the top portion of drawer 11. The mobile device is held by the magnetic force between the magnet attached to the back of the mobile device and the magnet attached to the top portion of drawer 11. According to another embodiment, a magnet is attached to the back of the mobile device and the top portion of drawer 11 comprises metal. The mobile device is held by the magnetic force between the magnet attached to the back of the mobile device and the metal on the top portion of drawer 11.

The system standing surface further comprises a cover 16 mounted on top of the main body 10. The recess 15 height (thickness) is such that when a mobile phone is mounted on the drawer, cover 16 will not touch the mobile device (and will be above it). The cover 16 is comprised of one or more of the following materials: pvc, Perspex, or any other polymer material and preferably Perspex. The cover 16 is attached to main body 10 preferably by means of screws or glue. The diameter of cover 16 is similar to that of main body 10 and its thickness is usually between 4 mm and 8 mm and preferably 6 mm.

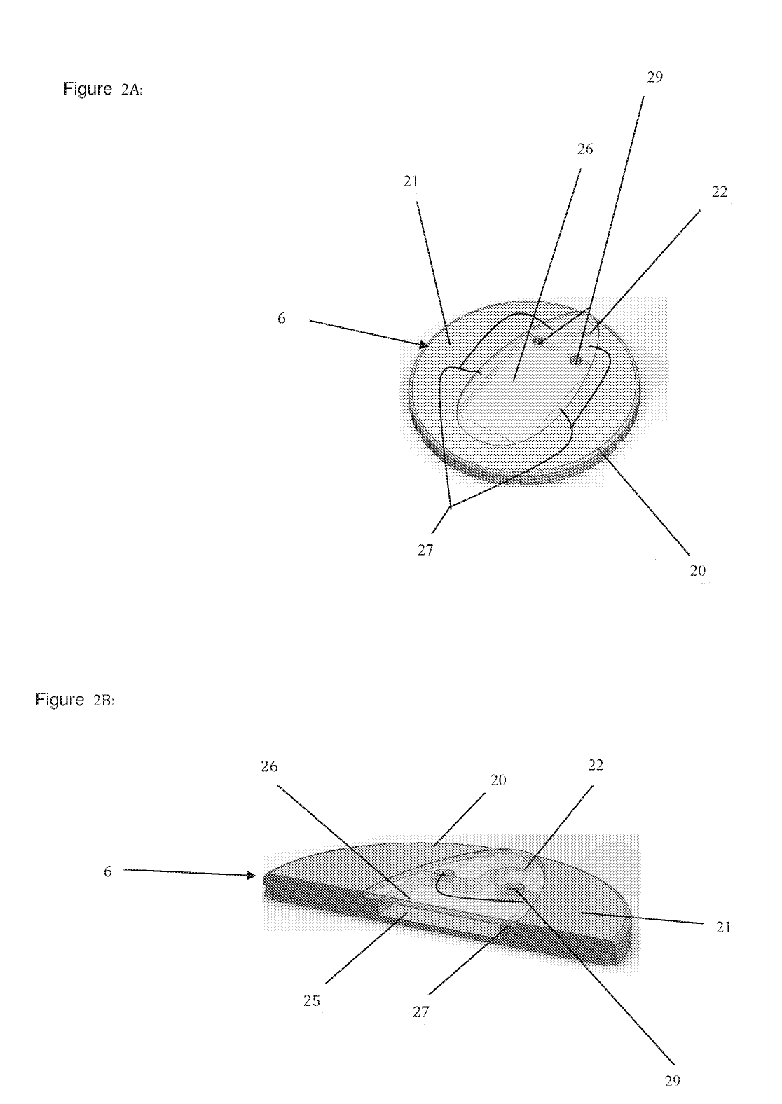

According to another embodiment of the present invention the mountable standing surface 6 (which is mounted onto an existing standing surface of an unstable balance surface) comprises a main body portion 20, as illustrated in FIGS. 2A and 2B. Main body portion 20 comprises a standing surface 21 comprising a removable cover 26 configured to cover a recess 25 within the main body portion 20. The top portion of removable cover 26 is leveled with the rest of the standing surface level. The circumference of the removable cover 26 is placed on leveled step portions 27 that surround recess 25. The height of the step portions 27 (from the bottom portion of recess 25 to top of step portions 27) is the same and is such that when the cover 26 is placed on them, it is leveled with the rest of the standing surface 21. The height of the step portions 27 is usually around 16 mm. The thickness of cover 26 is usually around 6 mm. The recess 25 area is big enough to receive a mobile device (e.g. 8 inches on 8 inches). The cover 26 can have various shapes and is preferably circular or oval (as in FIG. 2A). According to a preferred embodiment, cover 26 is transparent. In this way a user can control an application (e.g. play a game) while viewing the mobile device through cover 26. The cover 26 is preferably made of material selected from the group consisting of plastic Perspex, pvc, ocolon and polyetilen.

Preferably, the cover 26 comprises two finger apertures 29 configured for a user to insert a finger in each aperture 29 (e.g. thumb and index finger) for placing and removing cover 26. The apertures comprise a diameter usually between 1 cm and 3 cm and preferably 1.5 cm. Preferably, main body portion 20 comprises a groove between two step portions 27 configured to receive the cable connecting the mobile device to the external display means. Preferably, the groove has a twisted (snaking) configuration (as shown in FIGS. 2A and 2B) which assists in fixing the cable and preventing the cable from disconnecting from the mobile device and preventing unnecessary movement.

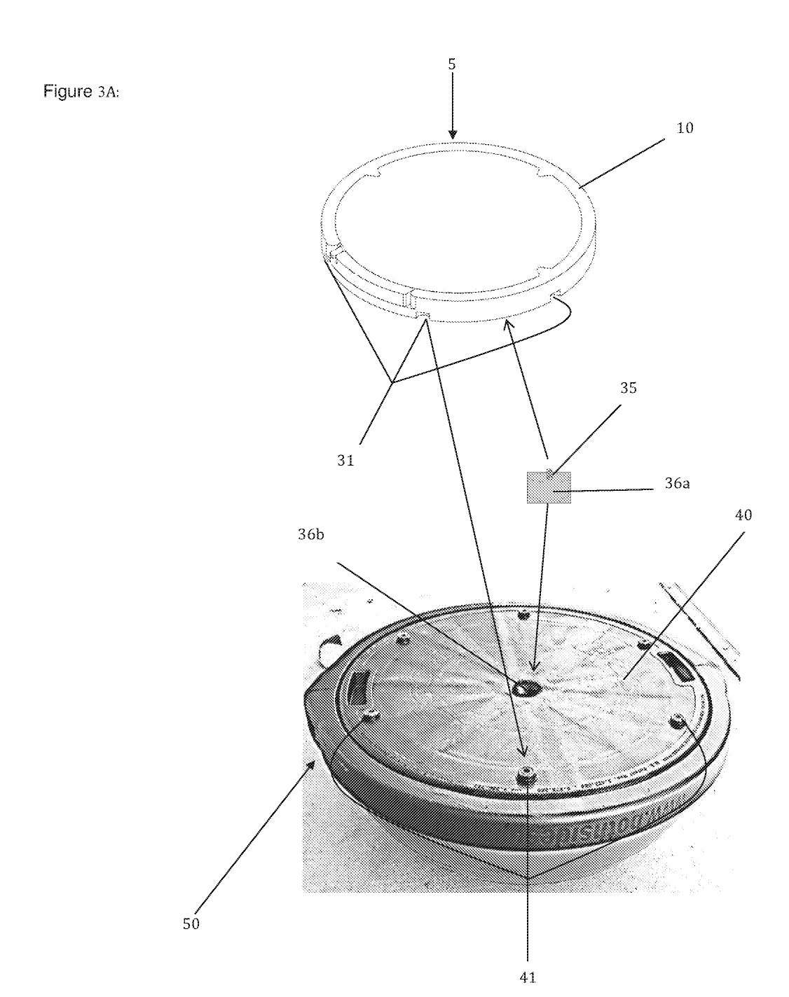

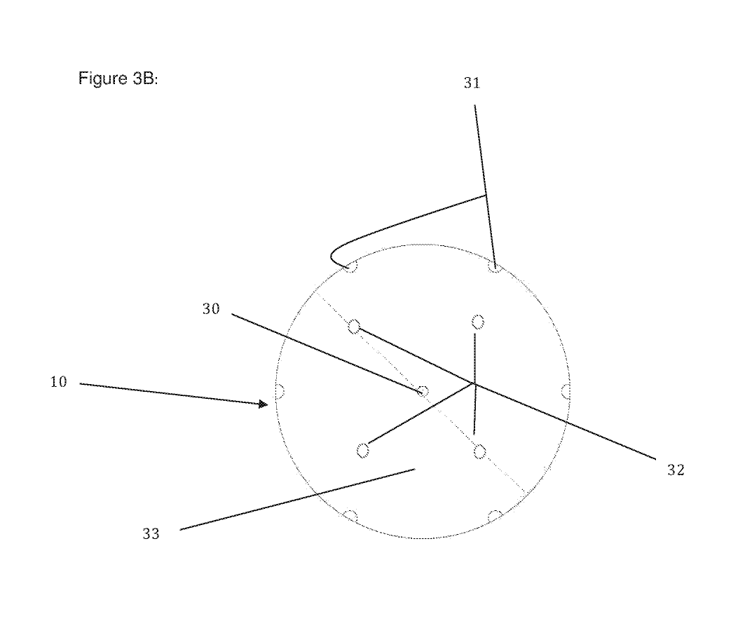

FIG. 3A shows an embodiment of the present invention with a basic standing surface 40 of an unstable balance surface 50 (BOSU) and a mountable standing surface 5 main body portion 10 configured to be mounted on the basic standing surface 40.

The standing surface 40 comprises a male connector 35 for fixing the main body portion 10 thereto. Optionally, the male connector 35 is connected to a base portion 36a that can be removed from the standing surface 40. The standing surface 40 comprises a recess 36b for receiving the base portion 36a (wherein the base portion 36a is complementary to the recess 36b). Preferably, the recess 36b placed at the center of the standing surface 40.

The diameter of base portion 36a is usually between 40 mm and 60 mm and its height is usually between 15 mm and 45 mm. Typically, recess 36b could be a typical inflation opening common in existing unstable balance surfaces

The main body portion 10 comprises a female component 30 on its bottom surface 33, as shown in FIG. 3B. The female component 30 is preferably placed at the center of the bottom surface 33. The female component 30 is configured to receive the male connector 35 in a fixed manner (e.g. by pin connection or by screw connection). The removable base portion 36a and the male connector 35 can be firstly connected to the female component 30 (and being fixed thereto) before the main body portion 10 is mounted on the standing surface 40. Then when mounted, the removable base portion 36a (connected by male connector 35 to bottom surface 33) is seated in the recess 36b, thus fixing the body portion 10 to the surface 40.

Preferably, for strengthening fixation, the standing surface 40 comprises a plurality of bulges 41 placed along its edge (or placed along the edge slightly inwards near the edge). Accordingly, bottom surface 33 comprises a plurality of recesses 31 complementary to the bulges 41, placed along the edge of the bottom surface 33 (or placed along the edge slightly inwards near the edge).

Preferably, the main body portion 10 comprises a plurality of female components 32 on its bottom surface 33, (preferably 4) as shown in FIG. 3B. The female components 32 are preferably placed and form the corners of a rectangle (preferably square), on the bottom surface 33. Preferably the center of the rectangle collides with the center of the bottom surface 33. The female components 32 are configured to receive a plurality of bulges placed on the standing surface, at a matching location. The female components 32 are complementary to the bulges, such that the mountable standing surface is fixed thereon.

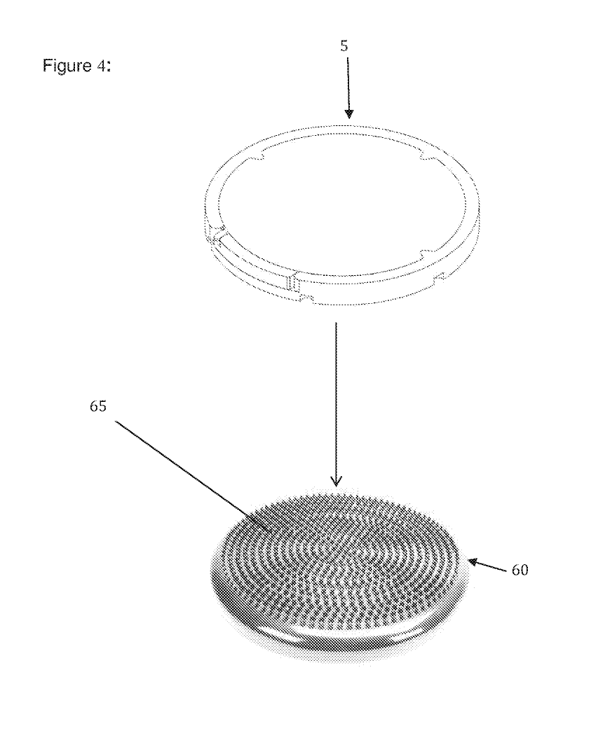

FIG. 4 illustrates an embodiment of the present invention with the mountable standing surface 5 mounted on a balance disc 60 having a coarse, rough top surface 65. The standing surface 5 according to this embodiment corresponds to the size of balance disc 60 preferably having similar diameters. When the mountable standing surface 5 is mounted on the balance disc 60 it is fixed due to the friction between the bottom surface 33 of mountable standing surface 5 and the coarse, rough top surface 65 of the balance disc 60. An example of such balance disc 60 is the AVITA balance disc catalog no. 59180.

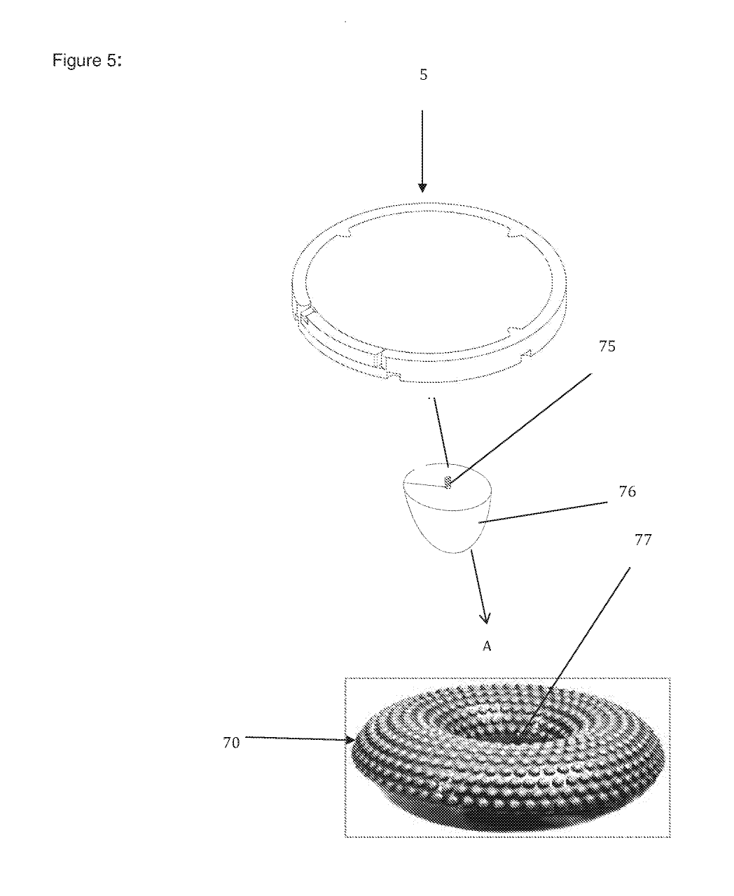

FIG. 5 illustrates an embodiment of the present invention with the mountable standing surface 5 mounted on a bagel balance disc 70. The standing surface 5 according to this embodiment corresponds to the size of bagel balance disc 70 preferably having similar diameters. When the mountable standing surface 5 is mounted on the bagel balance disc 70 it is fixed due to the friction and pressure between the bottom surface 33 of mountable standing surface 5 and the coarse, rough top portion of the bagel balance disc 70. Preferably, to strengthen fixation, a male connector 75 is connected to a half egg shaped base portion 76. The base portion 76 is complementary to the bagel balance disc 70 central hole 77. The male connector 75 is connected into the female component 30 on the bottom surface 33. When the standing surface 5 is mounted on bagel balance disc 70 the base portion 76, connected to surface 33, and complementary to the bagel balance disc 70 central hole 77 provides an efficient fixation. The diameter of the base portion 76 is usually between 5-9 cm and it height usually between 3-5 cm. An example of such balance disc is “the Abilitations Seat Cushion Balance Bagel” Model No. 0885634056803.

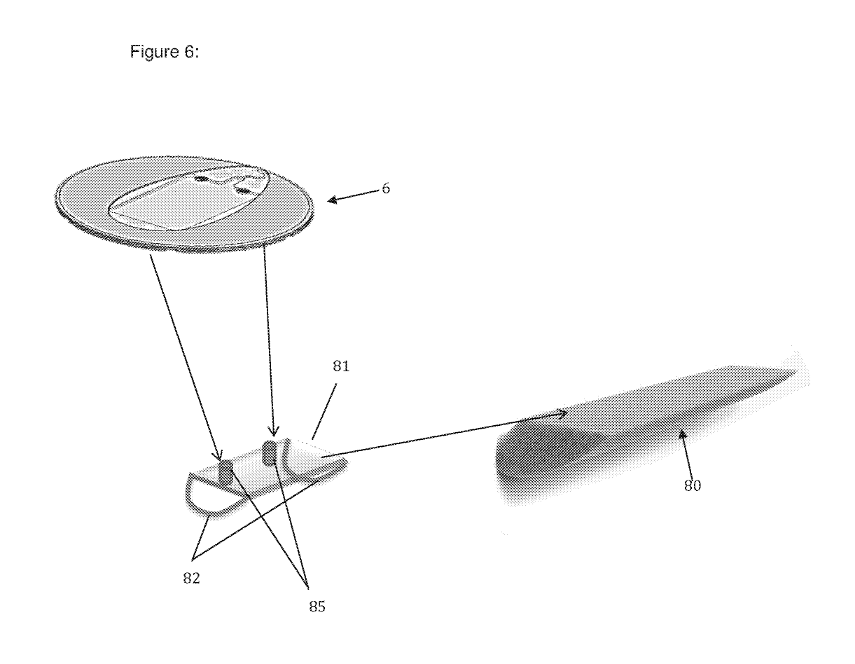

FIG. 6 illustrates an embodiment wherein mountable standing surface 6 is mounted on a half foam roller 80. According to one embodiment, a mountable surface 81 is mounted on the half foam roller 80, and attached and fixed thereto. Connecting means 82 such as rubber rings or straps fix the mountable surface 81 on to the half foam roller 80. Preferably, the mountable standing surface 6 main body portion comprises a plurality of female components on its bottom surface configured to receive a plurality of bulges or male connectors 85 placed on the mountable surface 81, at matching locations. The female components are complementary to the bulges or male connectors 85 such that the mountable standing surface 6 is fixable thereon. The length of the mountable surface 81 is usually between 30-50 cm. Its width is usually between 15-25 cm. Its thickness is usually between 5-15 mm.

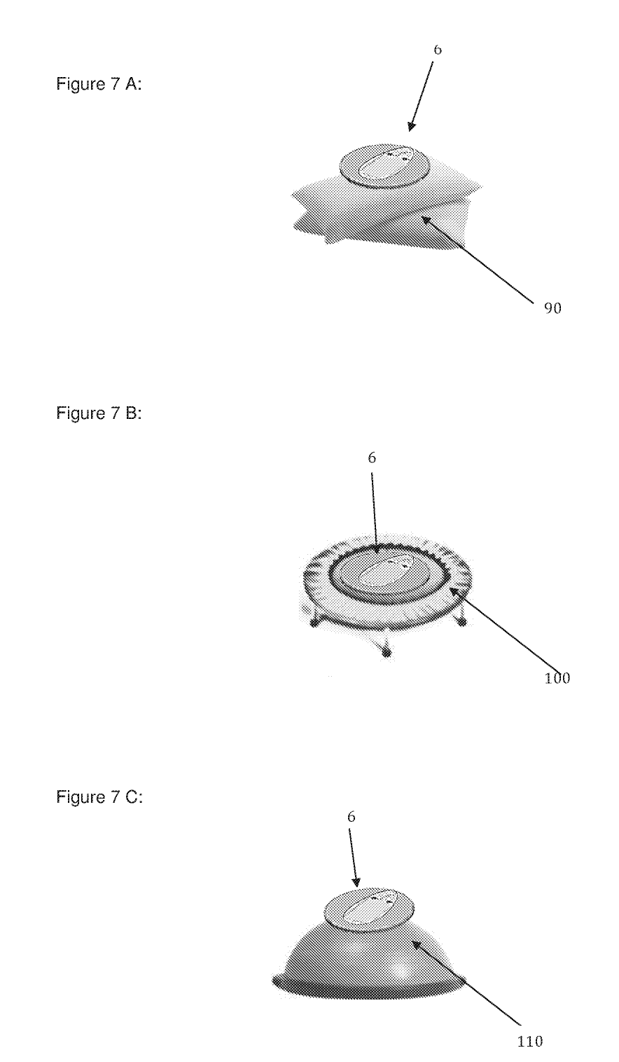

The present invention is very useful in which the mountable standing surface 6 is mountable on various platforms and items. FIG. 7a shows the mountable standing surface 6 being mounted on pillows 90. A user can use one or more of his standard pillows at home as a receiving platform for the mountable standing surface 6. The pillows 90 are used as a platform that enable the mountable standing surface 6 to tilt.

FIG. 7b shows the mountable standing surface 6 being mounted on a trampoline 100 (for example: Reebok 6 leg 91 cm Trampoline.122/6869EAN: 5055436101970). The trampoline 100 enables a user to tilt the standing surface 6, occasionally by applying additional pushing forces. This feature also may have additional physiotherapy advantages.

FIG. 7c shows the mountable standing surface 6 being mounted on an upside BOSU device 110 (mounted on the round portion). This feature also may have physiotherapy advantages, such as keeping balance, etc.

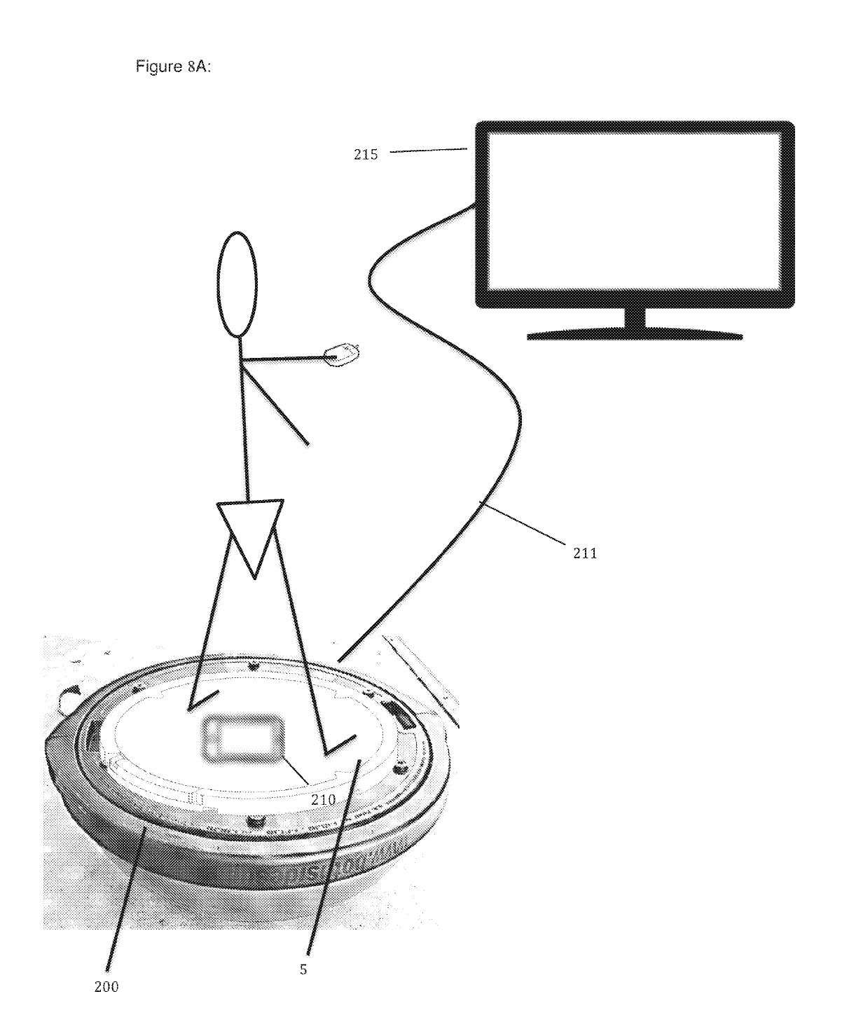

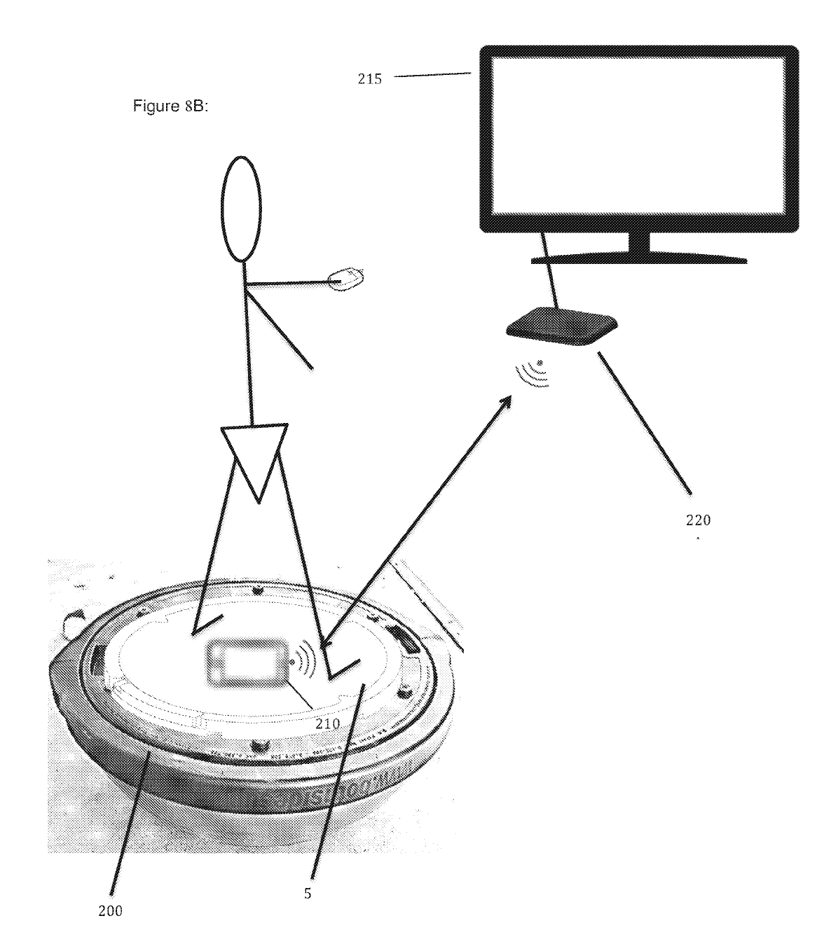

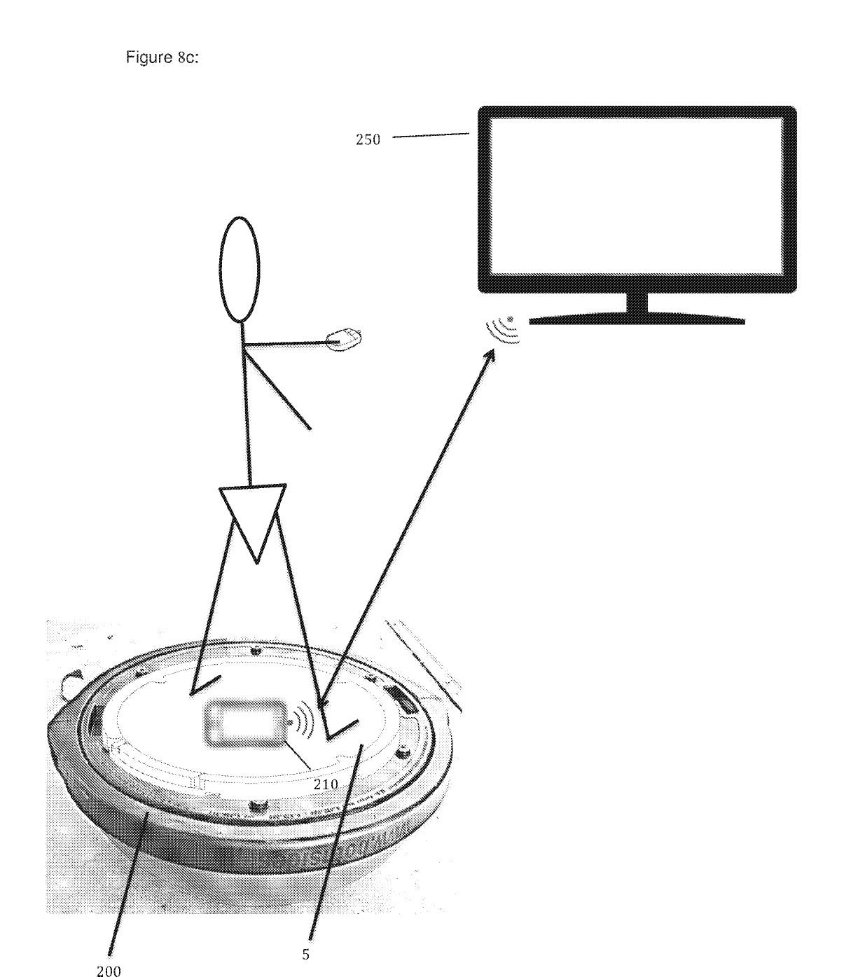

FIGS. 8A-8C illustrate examples involving an upside down BOSU device. FIG. 8A illustrates an example of a man standing on a mountable standing surface 5 mounted on a BOSU device 200. A mobile device 210 is fixed within the mountable standing surface 5. The mobile device 210 runs the application according to the changes in the gyroscopic/acceleration data, caused by the user's tilting. The mobile device 210 is connected by a wire connection 211 to a display means—screen 215. In this case the processor is within mobile device 210 and it runs the program.

FIG. 8B illustrates an example of a man standing on a mountable standing surface 5 mounted on a BOSU device 200. A mobile device 210 is fixed within the mountable standing surface 5. The mobile device 210 transmits gyroscopic/acceleration data signals (caused by the user's tilting) through wireless transmitting means, (e.g. mirroring technologies such as Apple's Air Server, Miracast, Bluetooth, WIFI, RF, etc.) to a receiving device 220, such as another mobile device (e.g. Tablet, Smartphone), a TV BOX, a DONGLE, a raspberry pi, all having an operating system. The receiving device 220 is connected (e.g. by wire) to the display means—screen 215. In this particular embodiment, the gyroscopic/acceleration data is wirelessly transferred to the receiving device 220 with the processor, which runs the application accordingly. The screen 215 shows the application running.

FIG. 8C illustrates an example of a man standing on a mountable standing surface 5 mounted on a BOSU device 200. A mobile device 210 is fixed within the mountable standing surface 5. The mobile device 210 transmits gyroscopic/acceleration data signals (caused by the user's tilting) through wireless transmitting means, (e.g. mirroring technologies such as Apple's Air Server, Miracast, Bluetooth, WIFI, RF, etc.) to a Smart TV 250. In this particular embodiment, the gyroscopic/acceleration data is wirelessly transferred to the Smart TV which comprises receiving means, a processor and an operating system that run the application accordingly. The Smart TV 250 obviously comprises a display screen that shows the application running. It can be seen in these FIGS. 8A-8C) that the user man is holding a mouse in his hand, enabling control of the application in conjunction with the tilting.

An example of an application game used with the system of the present invention is shown in FIG. 9 and relates to maneuvering a ball/circle 300 along a certain track bypassing obstacles 302. The user stands on the unstable balance surface near an external display means. The game is viewed on the external display means. The ball is maneuvered according to the user tilting the unstable balance surface. The sensors provide the mobile device processor with real-time altitude/longitude/latitude changes. The ball 300 moves accordingly. This is especially useful for certain physiotherapy treatments requiring the patient to tilt the unstable balance surface to a certain direction and to a certain degree. Obstacles 302 viewed on the display means are placed at certain locations on the display means forcing the user to tilt in a certain direction. The idea is to cause the user to apply a certain tilt thus treating a certain leg muscle. This tilt-muscle requirement is reflected in the application by an obstacle that needs to be bypassed. Several program application can be made and updated for dedicated treatments that include requiring the movement/stretch of certain muscles. The physiotherapist desires this for an effective treatment and the patient wants to succeed in the game. This provides the patient with extra motivation to do the exercises (i.e. succeed in the game).

Furthermore, medical personnel can review and analyze the treatment results either by printing the course of the game or printing a table with the sensor data transfiguration. Medical personnel can conclude whether to provide medicine, re-apply a certain treatment, emphasize on treating a certain muscle, etc., all according to the analysis of the sensor/game data. There are times that the patient can apply the treatment/game from a distant location to that of the medical personnel, since the executed data can be processed from a far or at a later date. Certain applications can include an alarm in case the sensor data reaches a certain threshold.

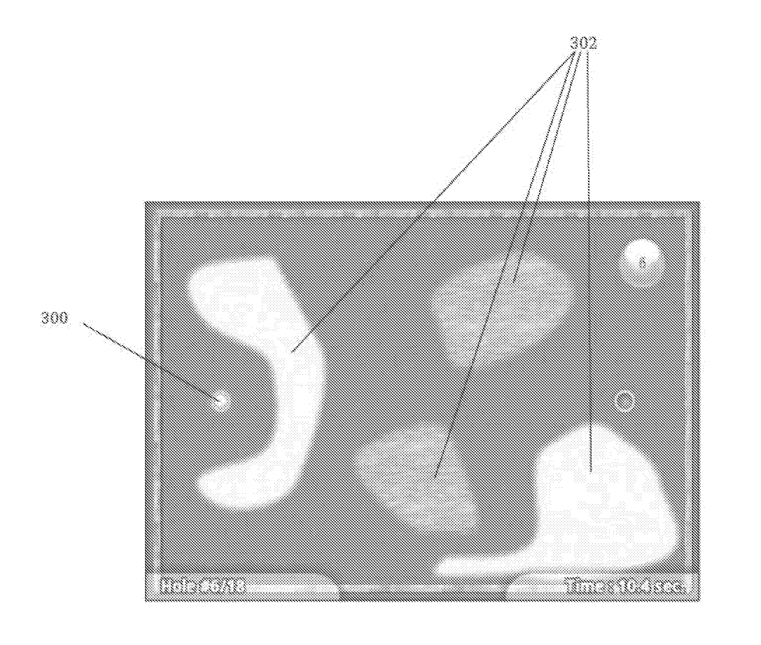

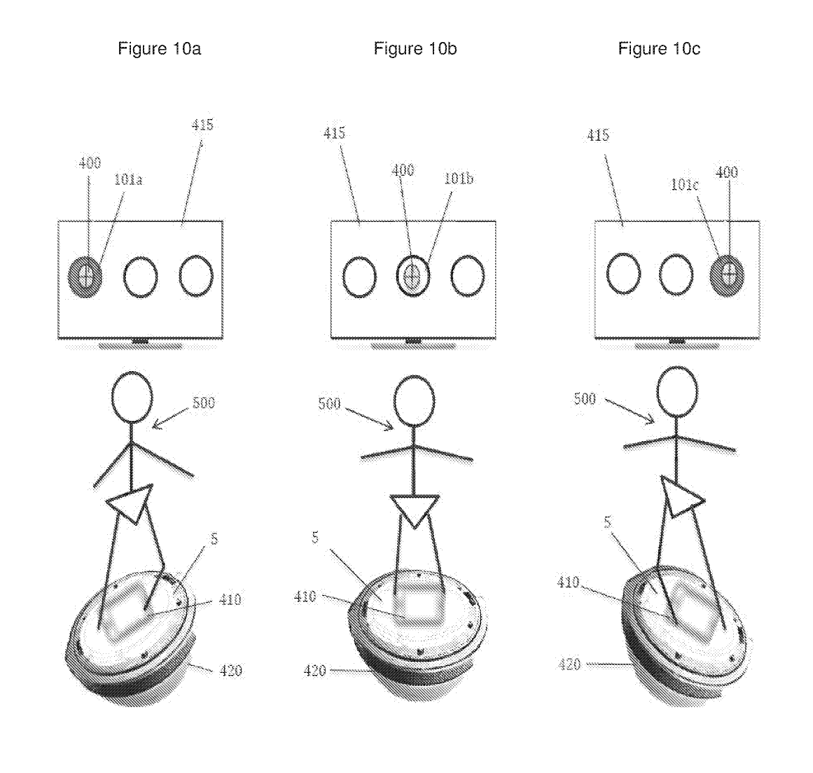

Another example of the game is shown in FIGS. 10a-10c with the mountable standing surface 5 mounted on a BOSU device 420. A mobile device 410 is fixed within the mountable standing surface 5. A display means 415 shows an application running. A circular sight 400 viewed on display means 415 moves according to the user's tilt. Accordingly the user patient must tilt to the direction such that the sight 400 will enter a certain circle. In FIG. 10b the sight 400 is within the centric circle 401b. The physiotherapist practitioner marks a certain circle. FIG. 10a shows a scenario where the practitioner has marked the left circle 101a. The user 500 then tilts the device leftwards. The sight accordingly moves leftwards to within the left circle 101a. FIG. 10c shows a scenario where the practitioner has marked the right circle 101c. The user 500 then tilts the device rightwards. The sight accordingly moves rightwards to within the right circle 101c.

The present invention further relates to a method comprising the following steps:
a. fixing accelerator or gyroscopic sensors within a mountable standing surface as explained herein (preferably fixing a mobile device comprising the accelerator or gyroscopic sensors);
b. mounting the mountable standing surface on a tiltable platform and fixing it thereto;
c. activating an application as explained herein;
d. tilting the mountable standing surface to control the application.

Preferably, the method is physiotherapy treatment method. Optionally, the method includes intervention in the application running by a physiotherapist practitioner.

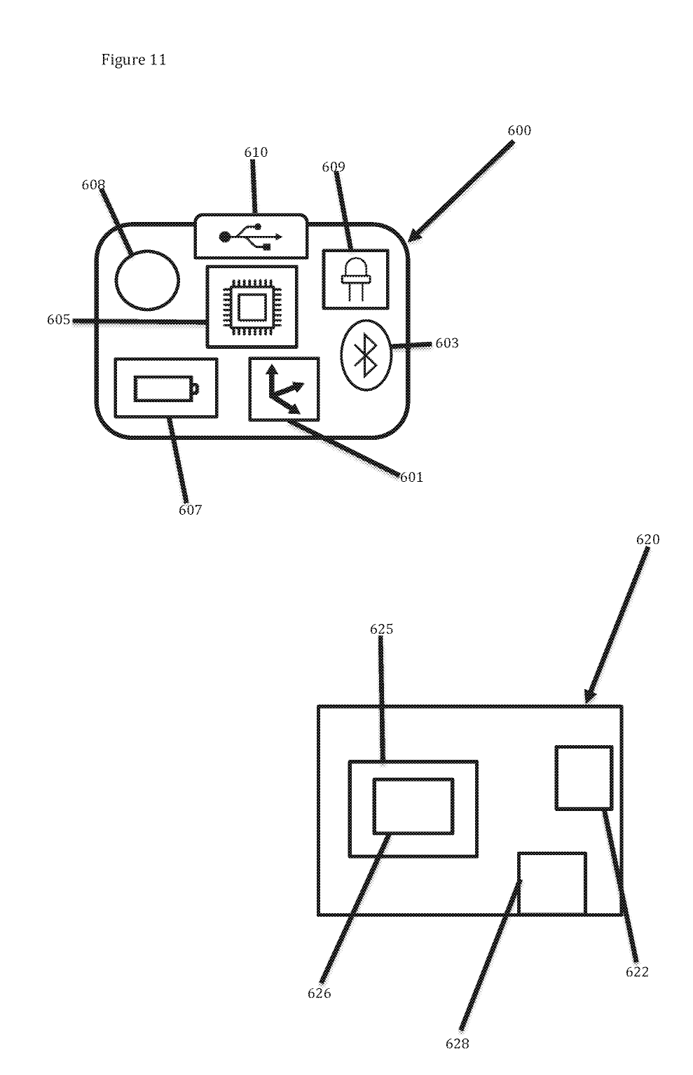

According to a very preferable embodiment, the present invention relates to an electronic device 600 (FIG. 11) mounted and fixed to a mountable standing surface. The electronic device 600 comprises a processor 605 (e.g. microprocessor or CPU) configured to receive location/position data and translate it (convert it) into a generic key stroke command of a standard keyboard (typically the arrow keys on the keyboard—forward (up), backward (down), right and left). These commands are transferred to a receiving unit of the game application processor (as will be described hereinafter) and used to control running game application items accordingly.

The electronic device comprises a tilting position sensor 601 preferably configured to calculate and provide position/movement/angle data (preferably in 3 axes—longitude, latitude and altitude) of the mountable standing surface (to which device 600 is fixed to). According to a preferred embodiment the tilting position sensor 601 is an accelerometer sensor. There is a correlation between the acceleration of the device 600 (and accordingly the mountable surface to which it is fixed thereto) and the angle (and movement) of the mountable standing surface at each axis. In a given event, as the acceleration exceeds in one direction, the angle increases accordingly. The microprocessor is programed such that it receives acceleration data and generates a corresponding (typically keyboard arrow) command. The acceleration data (at a certain axis) is transferred from the accelerator sensor to the processor 605 (to which it is coupled thereto) and converts the acceleration data to a corresponding keyboard command (according to a certain program).

The intensity of the acceleration data is translated into a corresponding command (typically causing moving of an item in a running application game). A high acceleration intensity causes a significant movement of the item. A low acceleration intensity causes a lower movement of the item. In this manner a “sensitive movable keyboard” if formed allowing operating/controlling keyboard command based applications.

According to the acceleration data, the processor 605 can estimate the angles of the tilt and generate a corresponding (typically keyboard arrow) command. For example, if the mountable standing surface is tilted forward—a forward arrow key command is generated. If the mountable standing surface is tilted backward or leftwards or rightwards—a backward or left or right arrow key command (respectively) is generated. Optionally, if the mountable standing surface is tilted to a diagonal direction (at a certain angle range, typically near degrees), two corresponding arrow key commands could be simultaneously generated (having a double event) correspondingly moving the application game item being controlled in a diagonal direction. The processor 605 converts the acceleration data to keyboard commands according to a certain defined protocol/algorithm—running on the processor 605.

Optionally, if an angle of the mountable standing surface tilts exceeding a certain threshold, the processor 605 generates a corresponding key command as if being constantly pressed as long as the threshold angle is exceeded. Optionally, at a certain angle range the processor can generate a corresponding command at a certain rate, and at a steeper angle range the rate is increased, etc., all according to the various application/game.

According to another embodiment the tilting position sensor 601 is a gyroscope (gyro) sensor. There is a correlation between the gyro data (angles/angular velocity) of the device 600 (and accordingly the mountable surface to which it is fixed thereto) and the angle (and movement) of the mountable standing surface at each axis. In a given event, as the gyro sensor data exceeds in one direction, the angle increases accordingly. The microprocessor is programed such that it receives gyro data and generates a corresponding (typically keyboard arrow) command. The gyro sensor data (at a certain axis) is transferred from the gyro sensor to the processor 605 (to which it is coupled thereto) and converts the gyro sensor data to a corresponding keyboard command (according to a certain program).

The intensity of the gyro sensor data is translated into a corresponding command (typically moving an item in a running application game). A high gyro sensor data intensity causes a significant movement of the item. A low gyro sensor data intensity causes a lower movement of the item. In this manner a “sensitive movable keyboard” if formed allowing operating/controlling keyboard command based applications.

According to the gyro sensor data, the processor 605 can estimate the angles of the tilt and generate a corresponding (typically arrow) command. For example, if the mountable standing surface is tilted forward—a forward arrow key command is generated. If the mountable standing surface is tilted backward or leftwards or rightwards—a backward or left or right arrow key command (respectively) is generated. Optionally, if the mountable standing surface is tilted to a diagonal direction (at a certain angle range, typically near 45 degrees), two corresponding arrow key commands could be simultaneously generated (having a double event) correspondingly moving the application game item being controlled in a diagonal direction. The processor 605 converts the gyro sensor data to keyboard commands according to a certain defined protocol/algorithm running on the processor 605.

According to both gyro sensor and accelerometer sensor data the corresponding arrow command can be generated according to a certain acceleration range or corresponding angle (gyro sensor data) range calculated. For example, if the mountable standing surface is tilted forward at an angle greater than 10 degrees, then the forward arrow key command is generated. Or if the acceleration data is in a certain range (or combinations thereof) then the corresponding key (or keys) command (typically an arrow key) is generated.

The device 600 further comprises a transmitter 603 configured to transmit the generated key stroke commands (generated by the processor 605) for controlling the application running. The transmitter 603 is coupled to the processor 605. The transmitter 603 may transmit according to Bluetooth, WIFI, RF, or other known wireless based communication protocols. The transmitter 603 may be a Bluetooth chip. The transmitter 603 receives the generated key command and transmits it to a remote device 620 (typically being in proximity to the user for viewing). Preferably, the keyboard data protocol is according to USB generic HID (Human Interface Device) standard protocol.

Typically, the accelerometer/gyroscope sensors (e.g. NORDIC sensors) data is according to a certain protocol (e.g. UART protocol) used for dedicated applications. The present invention processor 605 receives the accelerometer/gyroscope sensor data and converts it (by algorithm) to a generic keyboard protocol (HID).

The remote device 620 may be a smart TV, a PC, or most preferably a mobile device such as smart TV, a PC or most preferably, a mobile device such as an iPhone, iPad, and Galaxy etc. The remote device 620 comprises receiving means, such as a receiver 622 configured to receive the transmitted data. The remote device 620 further comprises a processor 625 and an operating system 626 that runs the game application. The received keyboard signals are transferred to the processor 625 running the application and the application (e.g. game) item is shifted according to the received command. The remote device comprises a display screen 628 that shows the application running.

The whole procedure is carried out in real time such that the user views an immediate change in the application which is a direct consequence to his tilting the mountable standing surface. The remote device 620 typically comprises means for receiving typical keyboard key commands (e.g. from a keyboard connected thereto by a keyboard interface connection means) for controlling applications, and instead is configured to transfer the present invention received keyboard stroke commands to the application operating system (running in the processor 625) for controlling the application. Transferring the received keyboard stroke commands (from device 600) to the remote device 620 operating system can be implemented by coupling the remote device receiver 622 output to a remote device keyboard interface input, or by directly transferring it to the operating system (e.g. by a dedicated application that is configured to do so).

The present invention is very advantageous, as it enables the electronic device 600 fixed to the mountable standing surface to control a large amount of application games, e.g. that are controlled by the arrow keys, and providing the user a more increased fun experience doing so. Also many applications that run on typical personal mobile devices that require, for example, forward, backward, left and right commands, can be controlled by the present invention. This advantage enables accessibility to a wide range of users that can simply download (e.g. from the internet for free) a wide range of application games (e.g. by downloading via google play store, Apple App store, etc) to their personal mobile devices and play the games with the present invention device 600 fixed to the mountable standing surface having a fun experience. In other words, the present invention enhances gaming experience of existing games accessible to a wide range of users.

The present invention also comprises an option for transmitting the location data as is for dedicated applications running on the remote device 620. These dedicated applications can control and shift an item in the dedicated application according to the location data (and not keyboard data) received. In this embodiment the location data from the location sensor 601 directly transfers the location data to the transmitter 603 (and not to processor 605) and is transmitted. Various dedicated applications (e.g. various games) read the location data and issue corresponding commands (e.g. shifting commands) according to the location data, each dedicated application according to its internal definitions.

The device 600 further comprises a powering means (for powering the device 600 elements) such as a battery or a rechargeable battery 607. The device 600 further comprises input means (such as buttons) for turning on the device. Optionally, the input means can be a button 608 for turning on the device 600 and choosing a using mode (i.e. transferring the location data to keyboard commands mode VS directly transferring the location data mode). The device 600 further comprises an interface for charging the rechargeable battery 607, preferably being a micro-USB port 610 (coupled to battery 607).

The device 600 advantageously further comprises indicating/illumination means such as an RGB LED 609. The various colors of flashing of the emitted RGB LED 609 can indicate several things. A non-limiting example is as follows: A green light indicates the transferring the location data to keyboard commands mode. A blue light indicates the directly transferring the location data mode (e.g. achieving this mode by two subsequent presses of button 608). A red light indicates low battery power. A flashing orange light indicates that the rechargeable battery 607 is currently charging. A constant orange light indicates that the battery 607 is full. Obviously, different colors/flashes can indicate these modes.

The device 600 length is usually between 20-50 mm, and preferably 30 mm. The device 600 width is usually between 20-40 mm, and preferably 27 mm. The device 600 thickness is usually between 2-15 mm, and preferably 7.5 mm. The device 600 external housing is preferably made of a material selected from the group consisting of plastic, rubber and any other polymeric material.

The mountable standing surface may be similar in size, general shape and material to that of the mountable standing surfaces as explained in detail hereinabove, with fixation means for fixing device 600 to it. It may be fixed by being placed in a dedicated sized pocket/recess (or drawer) comprised therein. The fixation may be carried out by sticking, by magnet, by friction, as explained hereinabove regarding the mobile device fixation, mutandis mutatis. Optionally the surface may be thinner than as explained hereinabove due to the smaller size of the fixing device 600.

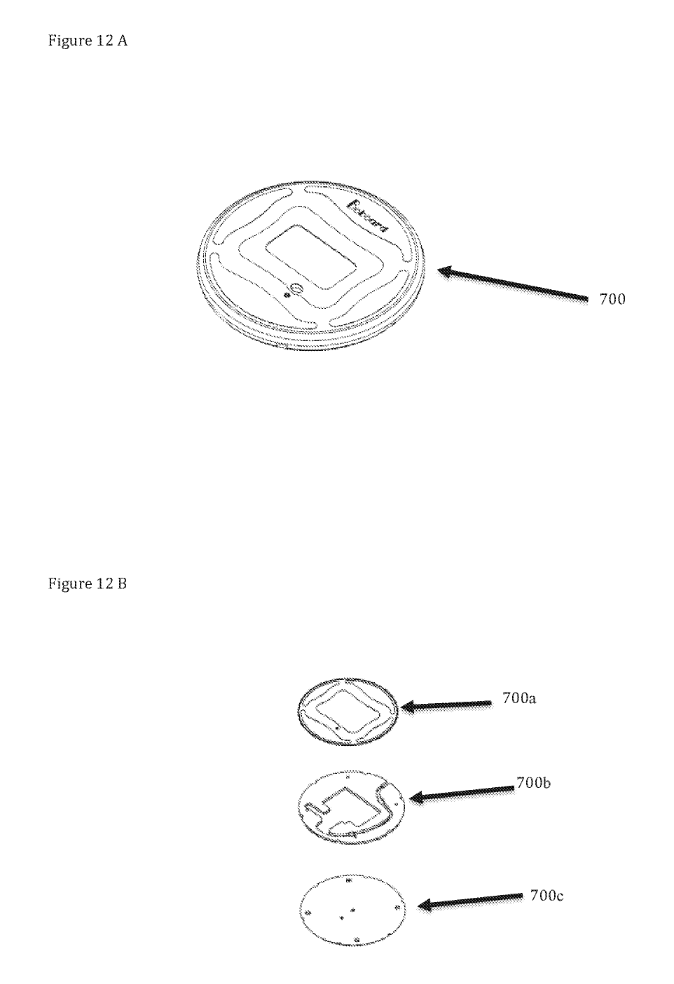

According to a specific embodiment of the present invention, the device 600 is fixed to mountable standing surfaces 700, shown in FIGS. 12a and 12b. The device 600 is connected to the remote device 620 by means of a cable (and thus the keyboard generated commands or the location data commands are transferred to the remote device 620 by the cable and not by a transmitter and are transferred to the remote device processor 625 and not to the receiver). The mountable standing surface 700 comprises a lid portion 700a attached to a middle portion 700b attached to a bottom portion 700c. FIG. 12b shows each portion separately. The mountable standing surface 700 middle portion 700b comprises a pocket portion 701 and a groove 702 having a twisted (snaking) configuration which assists in fixing the cable and preventing the cable from disconnecting from the device 600 and preventing unnecessary movement.

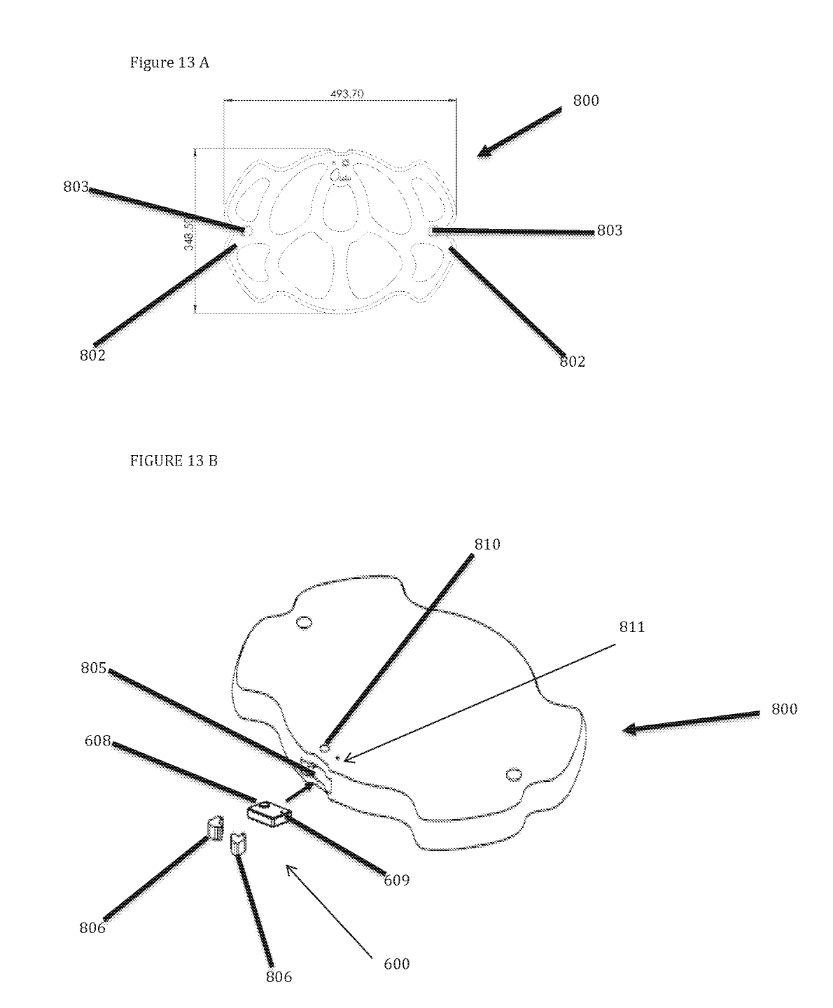

FIG. 13a shows a specific embodiment of the present invention wherein, the device 600 is fixed to mountable standing surface 800, shown in FIGS. 13a and 13b. Mountable standing surface 800 comprises wings 802 on its sides enabling additional space for the user's feet to stand on. The mountable standing surface 800 length is usually between 25-70 cm, and preferably 50 cm. The mountable standing surface 800 width is usually between 25-50 cm, and preferably 35 cm. The mountable standing surface 800 thickness is usually between 0.5-4 cm, and preferably 2 cm. Mountable standing surface 800 preferably comprises apertures 803 for bulges of the unstable balance surface to be inserted therein, thus providing an effective fixation. The apertures 803 diameters are fit such that the bulges tightly fit therethrough.

FIG. 13b shows the device 600 being inserted into a side recess 805 of mountable standing surface 800. The side recess 805 (or pocket) comprises dimensions such that the device 600 is fully inserted and fixed thereto. Optionally, support elements 806 are inserted for support of the device 600, assisting fixation and closure of the recess. The mountable standing surface 800 comprises an aperture 810 configured to be placed above the button 608 when the device 600 is fully inserted. Aperture 810 is fit in size and comprises a diameter for a user's finger to pass therethrough and press button 608 for activation. The mountable standing surface 800 comprises an aperture 811 configured to be placed above the RGB LED 609 when the device 600 is fully inserted. Aperture 811 is fit in size and comprises a diameter for a user to be able to view the RGB LED 609.

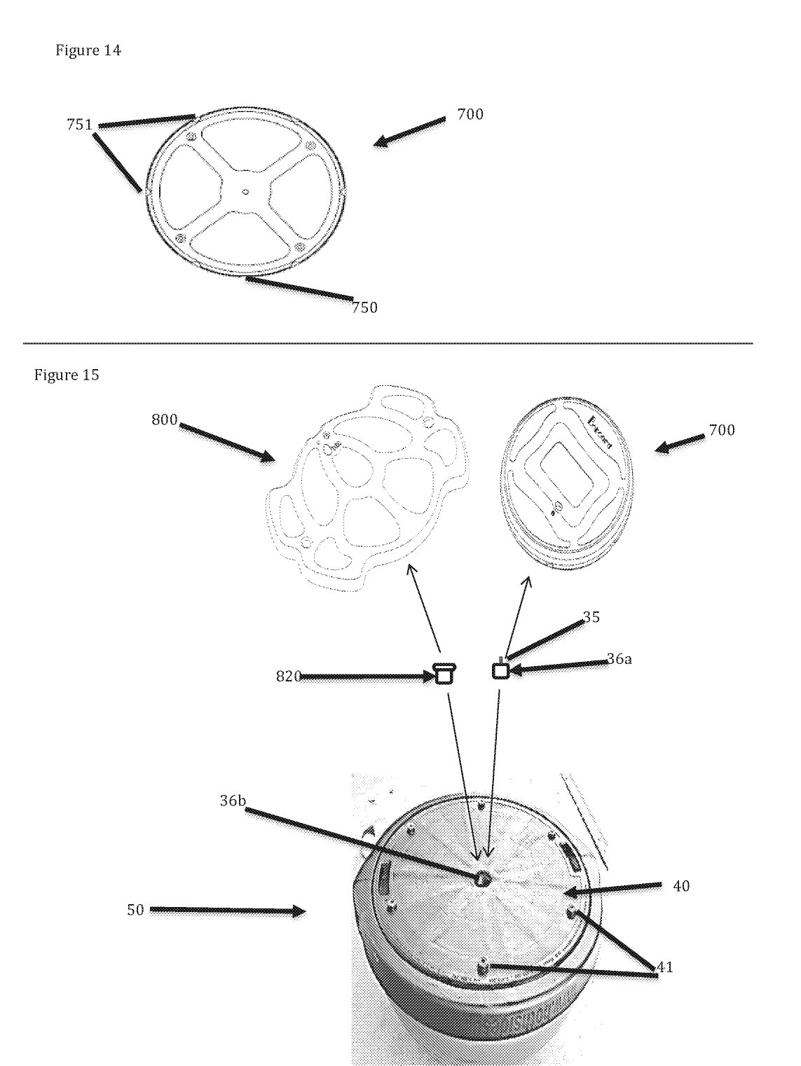

FIG. 14 shows mountable standing surface 700 with an opening 750 on its side at the end of groove 702 enabling the cable connected to the device 600 (according to the cable embodiment) to exit theretherough. Mountable standing surfaces 700 and 800 preferably comprise friction stickers stuck thereon on their top sides such that the friction stickers friction assists in fixation of a user's feet thereon. Mountable standing surfaces 700 and 800 preferably comprise friction stickers stuck thereon on their bottom sides such that the friction of the friction stickers assist in fixation of the mountable standing surfaces to top side of an unstable balance surface to which it is fixed thereon.

Optionally, the bottom surfaces of the mountable standing surfaces 700 and 800 comprise a plurality of recesses 751 (similar to elements 31 as described hereinabove) on there edges, complementary to the bulges of an unstable balance surface (similar to element 41 as described hereinabove). placed along the edges of the bottom surfaces of the mountable standing surfaces 700 and 800 (or placed along the edges slightly inwards near the edges).

FIG. 15 shows that the mountable standing surfaces 700 and 800 can connect to an unstable balance surface 50 (e.g. a BOSU, with elements 35, 36a, 36b, 40, 41 as explained hereinabove) in a similar manner as explained regarding the connection of the mountable surface board 5 to unstable balance surface 50 (e.g. with similar bulges recesses/holes for fixation), mutatis mutandis. It should be noted that mountable standing surfaces 700 and 800 comprising the device 600 can be mounted on and fixed to all the surfaces (e.g. standing surfaces of unstable balance surfaces or tiltable platforms) as explained hereinabove regarding the other mountable standing surface, mutatis mutandis.

Optionally, fixation of the Mountable standing surfaces 700 and 800 to the unstable balance surfaces may be implemented by a connector 820 which its top end is fit in shape to tightly fit into a complementary shaped aperture of the bottom surface of the Mountable standing surfaces 700 and 800. The connector 820 bottom end is fit in shape to tightly fit into a complementary shaped aperture of the top standing surface of the unstable balance surface.

According to an embodiment of the present invention device 600 is fixed to the standing surface of an unstable surface board (e.g. BOSO) by means of sticking, friction, dedicated recess/pocket, etc.

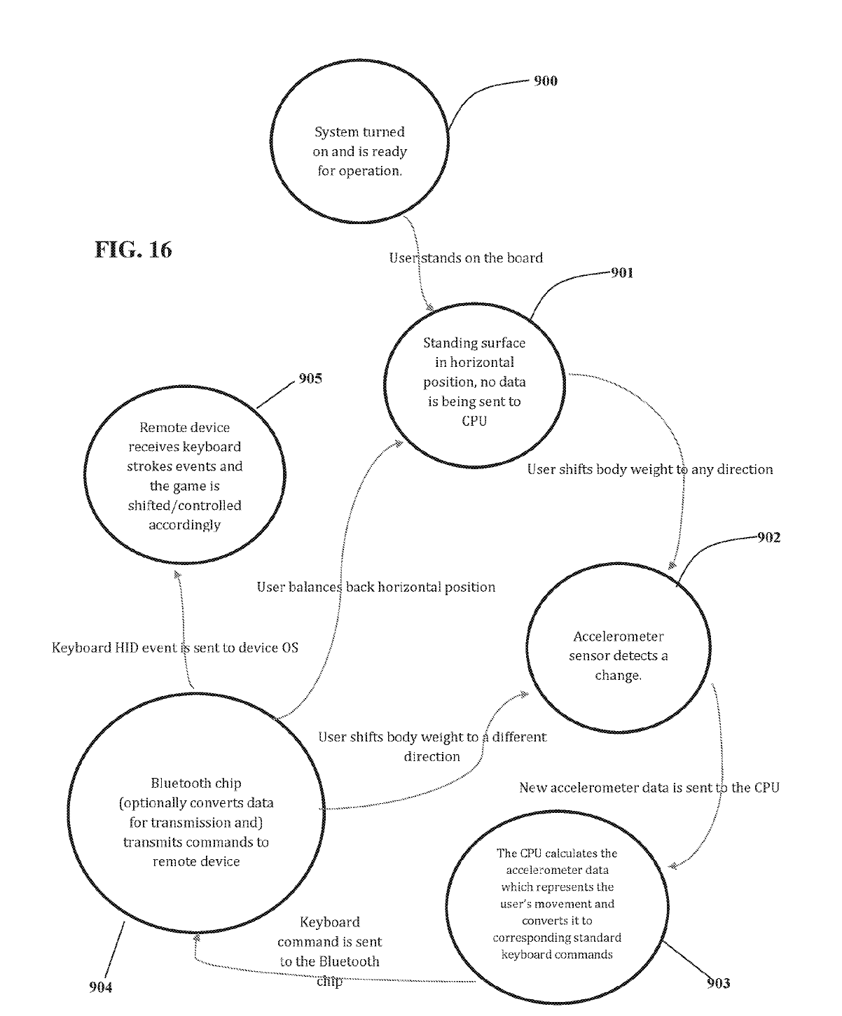

The present invention relates to a method for operating/controlling an application running on an operating system, according to the tilts of a standing surface, as shown in FIG. 16. This embodiment is explained in regards to an accelerometer sensor by can be executed with a gyro sensor mutatis mutandis. The transmitter is a Bluetooth transmitter and the processor is a CPU. Device 600 is fixed to a tiltable standing surface (e.g. mountable standing surface 700 or 800 mounted on an unstable balance surface). First (900), the system is turned on and is ready for operation. Then a user stands on the standing surface (the surface board, used herein interchangeably). When the standing surface is in horizontal position (901), no data is being sent to the processor (CPU) of device 600. The user shifts body weight to any direction on the standing surface. The accelerometer sensor detects a change (902) and new accelerometer data is sent to the processor. The processor calculates the accelerometer data (e.g. movement velocity and the board's tilt angle) which represents the user's movement and converts it to corresponding standard keyboard commands (903). The commands are sent to a transmitter, e.g. a Bluetooth chip (optionally converting the command data to a specific protocol (HID) for transmission and identifies the commands being transmitted as HID keyboard commands). The command (keyboard HID event) is transmitted to a remote device OS (904) which receives the keyboard strokes commands and the game is shifted/controlled accordingly (905). Simultaneously, the standing surface is in horizontal position again (901) or is tilted again and the accelerometer detects the change (902) and continues as explained.

The present invention relates to a method for controlling an application running on an operating system, according to the tilts of a standing surface, said method comprises the following steps:

providing a processor and a sensor,
a. obtaining accelerometer or gyroscope data from said sensor;
b. transferring said accelerometer or gyroscope data to a processor;
c. converting said accelerometer or gyroscope data into corresponding keyboard commands by said processor;
d. transmitting said keyboard commands to a remote device having a second processor running an application on an operating system;
e. transferring the transmitted keyboard commands to said running application to be controlled accordingly.

It should be noted that the mountable standing surface, the electric/electronic devices as explained herein can comprise or be used with other portions explained herein regarding different embodiments, mutatis mutandis.

While some of the embodiments of the invention have been described by way of illustration, it will be apparent that the invention can be carried into practice with many modifications, variations and adaptations, and with the use of numerous equivalents or alternative solutions that are within the scope of a person skilled in the art, without departing from the spirit of the invention, or the scope of the claims.

We claim:

1. A balance training system, comprising:
a. a standing surface mountable onto an unstable platform;
b. an electronic sensor device integrated into the surface, the sensor device comprising a tilt sensor providing tilt sensor data, a power source, a processor, and a data transmitter; and
c. a computing device for running multiple tilt-based applications, wherein the computing device uses the tilt sensor data exclusively of the sensor device to apply to the tilt based applications to apply a user's balancing behavior, wherein the sensor device is in communication with the computing device.

2. The system of claim 1, wherein the standing surface includes one or more fixing means on its bottom surface selected from the group consisting of sticking, friction, placing in a dedicated recess, and placing in a dedicated pocket.

3. The system of claim 1, wherein the unstable platform includes one or more surfaces selected from the group consisting of balancing balls, rollers, surfaces, and disks.

4. The system of claim 1, wherein the standing surface is adapted to be fixable on substantially any unstable platform.

5. The system of claim 1, wherein the tilt sensor includes one or more sensors selected from the group consisting of an accelerometers and gyroscopes.

6. The system of claim 1, wherein the data transmission means includes one or more transmission means selected from the group consisting of cables, mirroring technologies, sir server, miracast, Wi-Fi, Bluetooth, WIFI, and RF.

7. The system of claim 1, further comprising display means selected from the group consisting of a smart TV, PC, mobile device screen, TV Box screen, and projector.

8. The system of claim 1, wherein the training application includes code for applying user balancing behavior to one or more gaming applications to enable usage of such gaming applications as training applications.

9. The system of claim 1, wherein the user's balancing behavior is used to perform one or more training methods selected from the group consisting of physical exercises, training games, physiotherapy and neuromuscular training.

10. The system of claim 1, wherein the processor translates accelerometer data into keyboard strokes to facilitate playing multiple applications using a generic interface device.

11. A method for balance training, comprising:
fixing an electronic sensor device to a standing surface;
mounting the standing surface onto an unstable platform;
pairing the electronic sensor device having a tilting sensor with a remote computing device;
sensing a user's balancing behavior using the tilting sensor in the sensing device;
transmitting the user's balancing behavior data to the remote computing device; and
applying the user's balancing behavior data to one or more applications.

12. The method of claim 11, further comprising using the one or more applications as training applications.

13. The method of claim 11, wherein the transmitting of balancing behavior data includes transmitting substantially raw accelerator data to the computing device via UART protocol.

14. The method of claim 11, wherein the transmitting of balancing behavior data includes translating accelerometer data into key strokes, to facilitate playing multiple applications using a generic Human interface device (HID).

15. The method of claim 11, further comprising displaying the user's balancing behavior data in the one or more applications using a connected display means.

16. The method of claim 11, further comprising operating multiple tilt related applications with data exclusively from the tilting sensor in the sensing device.

17. The method of claim 11, further comprising converting a gaming application into a training application by applying the electronic sensor device data to a gaming application being operated by a user.

18. A method for tilt based gaming, comprising:
fixing an electronic tilt sensor device to a standing surface;
mounting the standing surface onto an unstable platform;
connecting the electronic device to an external computing device OS;
monitoring for changes in tilting position of the electronic sensor devices;
sending new accelerator data to the electronic sensor device processor;
calculating the movement velocity and the tilt angle of the unstable platform, to represent the user's movement;
transmitting calculated user movement data to a communications chip on the electronic sensor device;
translating the user movement data to keyboard strokes;
transmitting keyboard stroke data to the computing device OS; and
operating one or more tilt based games using the keyboard strokes.
